(19) United States
(12) Patent Application Publication
(10) Pub. No.: US 2007/0184510 A1
(43) Pub. Date: Aug. 9, 2007

(54) Method and apparatus for monitoring enzyme mixtures

(76) Inventors: Frederick Rowell, Sedgefield (GB); Latha Sundar, Sedgefield (GB)

Correspondence Address: PATENT ADMINISTRATOR;KATTEN MUCHIN ROSENMAN LLP, 1025 THOMAS JEFFERSON STREET, N.W., EAST LOBBY: SUITE 700, WASHINGTON, DC, 20007-5201, US

(21) Appl. No.: 11/349,261

(22) Filed: Feb. 8, 2006

(51) Int. Cl. C12Q 1/34; C12Q 1/37; C12M 3/00

(52) U.S. Cl. 435/18; 435/23; 435/287.1

(57) ABSTRACT

The present invention provides a method for simultaneously detecting the presence of at least two enzymes in a sample. The method includes the steps of: i) providing a first substrate for a first enzyme, the first substrate being labeled with a first fluorophore; ii) providing a second substrate for a second enzyme, the second substrate being labeled with a second fluorophore; iii) exposing the labeled substrates to the sample to allow the first and second enzymes present in the sample to interact with respective first and second fluorophore-labeled substrates to form respective first and second fluorophore-labeled substrate fragments; and iv) detecting the presence of the fluorophore-labeled substrate fragments.

### BACKGROUND

1. Field of the Invention

The present invention is concerned with monitoring for the presence and/or determining the concentration of mixtures of enzymes, and, in particular, makes possible such monitoring of enzyme mixtures on a continuous basis.

2. Background Information

Enzymes of various types are widely used throughout a variety of industries, such as, for example, the pharmaceutical industry, biotechnology industry, soap and detergent industry, the food industry and the like. It has been observed that inhalation of enzymes released into the workplace atmosphere can cause deleterious health effects on exposed workers resulting from respiratory sensitization. Statutory exposure limits have been identified, such as for the proteolytic enzyme subtilisin and related enzymes, and it is a statutory requirement that the work place exposure limit (WEL) of 40 ng/m³ for this class of enzyme is enforced, as described in, for example, EH40/2005 Workplace exposure limits, Environmental Hygiene Guidance Note EH40, HSE Books, ISBN 07176 29775. In order to conform to this requirement, it is necessary to monitor for exposure at regular intervals or on a continuous basis for extended periods.

Systems for monitoring airborne enzymes currently consist of capturing the enzyme from the air, extracting the captured enzyme into solution and analyzing the resulting solution for the constituent enzymes. Conventional methods for capture consist of passing air though a filter, and analysis is largely based on spectrophorometric or spectrofluorescent methods in which the enzyme hydrolyses molecules of labeled substrate in solution, as described in, for example, Rothgeb, T. M., Goodlander, B. D., Garrison, P. H. & Smith, L. A., J. Am. Oil Chemists Soc., 65, p. 806 (1988), or in an immunoassay format such as ELISA, as described in, for example, Miao, Z-H, Rowell, F. J., Reeve, R. N., Cumming, R. H., Journal of Environmental Monitoring, 2, pp. 451-454 (2000). The combination of capture onto filters followed by complex extraction and analysis cannot be developed to produce a sensitive, reagentless and continuous method for near real-time monitoring of airborne enzymes, and the results obtained represent time averaged values, since intermittent release of enzymes cannot be monitored.

A method has been described that combines capture from air via impaction onto the surface of a cyclone and immediate analysis of the captured enzyme from the washed surface of the cyclone. The latter is achieved using fluorescent-labeled substrate specific for the enzyme in question that is immobilized onto a solid phase support contained within a fixed bed or bioreactor. Passage of the enzyme though the bioreactor results in partial digestion of the substrate and detection of the fluorescently-labeled fragments downstream, as described in, for example, International Application No. PCT/GB96/03052, filed Dec. 11, 1996. This system allows continuous and near real time monitoring of single enzymes in the atmosphere, as described in, for example, Tang, L. X., Rowell, F. J., Cumming, R. H., Annals Occupational Hygiene, 40, pp. 381-389 (1996).

To date, no system has been described that can simultaneously monitor mixtures of airborne enzymes on a continuous basis and in near real-time. It is the object of the present invention to provide a method for monitoring enzyme mixtures for use on a continuous basis or for monitoring at frequent intervals.

### SUMMARY OF THE INVENTION

The method herein described according to exemplary embodiments of the present invention enables the simultaneous qualitative and/or quantitative monitoring of a sample for the presence of at least two enzymes to be undertaken.

According to an aspect of the invention, there is provided a method for the simultaneous detection of at least two enzymes in a sample, the method comprising the steps of:
i) providing a first substrate for a first enzyme, the first substrate being labeled with a first fluorophore,
ii) providing a second substrate for a second enzyme, the second substrate being labeled with a second fluorophore,
iii) exposing the labeled substrates to the sample to allow the first and second enzymes present in the sample to interact with respective first and second fluorophore-labeled substrates to form respective first and second fluorophore-labeled substrate fragments; and
iv) detecting the presence of the fluorophore-labeled substrate fragments.

The substrates used in the method are selected from among those with which the enzyme to be monitored reacts/digests.

In a preferred embodiment of the present invention, the substrate can be a protein or polypeptide. Preferably, the protein is selected from the group including, for example, gelatin, porcine thyroglobulin, collagen, immunoglobulin, bovine serum albumin and the like.

The labeled substrates are, in many cases, advantageously carried upon a suitable support, such as, for example, glass beads, fibrous cellulose or sol gel particles. The particle size can range from approximately 100 nm to approximately 100 μm in diameter, although the particles can be of any suitable size.

In a further preferred embodiment of the present invention, the beads are magnetizable.

Examples of enzyme types that can be detected by the application of the method according to exemplary embodiments include, but are not restricted to, members of the protease, cellulase, lipase, amylase or collagenase families.

Even more preferably, the protease is selected from the group including, for example, a subtilisin-type enzyme, trypsin, papain, esperase, alcalase or the like.

In a preferred embodiment of the present invention, the first substrate can be gelatin and the first enzyme can be a subtilisin-type enzyme, while the second substrate can be starch, amylase, amylopectin or the like, and the second enzyme can be, for example, α-amylase.

The fluorophore used in the method of the present invention to label the substrates is selected to be readily and consistently detectable by normal fluorometric methods and chosen so that its spectral properties do not interfere with the fluorescent signals of other fluorophores co-released via the reaction of other enzymes present in the mixture with their specific substrates. Preferably, the first and second fluorophore is selected from the group including fluorescein, rhodamine, TEXAS RED™ or lucifer yellow.

The labeled fragments released from each vessel following exposure to the sample containing the enzymes are monitored downstream using spectrofluorimeter(s) tuned to a specific fluorophore.

In a preferred application of the method according to exemplary embodiments of the present invention, the sample can be, for example, air. A particularly useful application of the method is in the work place, where there are statutory limits of employees' exposure to air-borne enzymes.

The method can be put into practice in various ways and manners, but it is preferred to practice it on a continuous basis by conveying the sample, for example air, that contains the enzymes continuously into a collector containing a solution that then contacts the labeled substrates in a vessel.

The preferred solution is an aqueous buffer, for example phosphate buffered saline or the like. A detergent, such as, for example, Tween 20 or the like, can be utilized to reduce non-specific binding between released fragments and surfaces within the flow injection analysis system.

In a preferred embodiment of the method of present invention, the substrates can be provided in a reaction vessel such that the enzyme(s) derived from the sample simultaneously contact the first substrate and the second substrate. For example, where the substrates are supported on beads, the reaction vessel can contain a heterogeneous mixture of the beads.

In an alternative exemplary embodiment of method of the present invention, the enzyme(s) derived from the sample sequentially contact the first substrate and the second substrate. For example, the reaction vessel can contain stratified homogeneous layers of the different substrates.

In a further alternative exemplary embodiment of method, two reaction vessels can be positioned in series, with a conduit for the sample being provided between the two vessels. In such an exemplary embodiment of the present invention, the first vessel can contain the first substrate and the second vessel can contain the second substrate.

When it is expected that the sample being analyzed contains a protease, it is preferable to ensure that the sample contacts the substrates sequentially. Even more preferably, the substrate for the protease can be positioned adjacent to the detecting means for optimal performance of the method. It has been found that fragments of the digested protease substrate stick to the solid phase material supporting the other substrate(s). For example, when detecting α-amylase and a subtilisin-type enzyme, the substrate for the subtilisin-type enzyme is preferably positioned adjacent to the detecting means.

The reaction vessel(s) can be, for example, a column. The reaction vessel(s) are preferably small and can be readily carried to, or installed at the site to be monitored.

The detection of the fluorescently-labeled substrate fragment can be undertaken on site (e.g., at a factory), if the appropriate equipment, for example, a spectrophotometer or the like, is available. Alternatively, the reaction vessel can be taken to a laboratory where the analysis is undertaken.

According to a further aspect of the present invention, there is provided a vessel for use in the simultaneous detection of at least two enzymes in a sample. The vessel includes a first substrate for a first enzyme, the first substrate being labeled with a first fluorophore. The vessel also includes a second substrate for a second enzyme, the second substrate being labeled with a second fluorophore.

In a preferred embodiment of the present invention, the labeled substrates are carried upon a suitable support, for example, glass beads, fibrous cellulose, sol gel particles or the like. The particle size can range from approximately 100 nm to approximately, 100 μm in diameter, although the particles can be of any suitable size.

In an exemplary embodiment of the present invention, the reaction vessel can contain a heterogeneous mixture of the beads. In an alternative exemplary embodiment of the present invention, the reaction vessel can contain stratified homogenous layers of the substrates.

According to a further aspect of the present invention, there is provided an apparatus for use in the simultaneous detection of at least two enzymes in a sample. The apparatus includes a vessel that includes a first substrate for a first enzyme, the substrate being labeled with a first fluorophore, and a second substrate for a second enzyme, the substrate being labeled with a second fluorophore. The apparatus also includes detecting means for detecting fluorescent reaction products.

Preferably, the first and second substrates can form stratified layers within the vessel and the second substrate, being a substrate for a protease, can be located adjacent to the detecting means.

The detecting means can be any suitable means for detecting. For example, the detecting means can be a spectrophotometer or the like.

According to a still further aspect of the present invention, there is provided an apparatus for use in the simultaneous detection of at least two enzymes in a sample. The apparatus includes a first vessel that includes a first substrate for a first enzyme, the substrate being labeled with a first fluorophore, and the first apparatus being connectable to a second vessel. The second vessel includes a second substrate for a second enzyme, the substrate being labeled with a second fluorophore. The apparatus also includes detecting means for detecting fluorescent reaction products.

In a preferred embodiment of the apparatus according to an exemplary embodiment of the present invention, the substrate in the second vessel can be a protease or the like, and the second vessel can be located adjacent to the detecting means. The detecting means can be any suitable means for detecting. For example, the detecting means can be a spectrophotometer or the like.

### BRIEF DESCRIPTION OF THE DRAWINGS

Other objects and advantages of the present invention will become apparent to those skilled in the art upon reading the following detailed description of preferred embodiments, in conjunction with the accompanying drawings, wherein like reference numerals have been used to designate like elements, and wherein:

FIG. 1A illustrates a calibration plot for subtilisin-type protease (SAVINASE™) enzyme alone, in accordance with an exemplary embodiment of the present invention.

FIG. 1B illustrates a calibration plot for an α-amylase enzyme (TERMAMYL™) alone, in accordance with an exemplary embodiment of the present invention.

FIG. 1C illustrates a calibration plot for α-amylase in an equi-concentration mixture of the subtilisin-type protease and α-amylase, in accordance with an exemplary embodiment of the present invention.

### DETAILED DESCRIPTION OF THE PREFERRED EMBODIMENTS

Sol gel particles activated with glycidoxypropyltrimethoxysilane were treated with gelatin pre-labeled with TEXAS RED™ to produce a solid phase substrate-coated solid phase support for subtilisin-type proteases (e.g., SAVINASE™ or the like).

Other sol gel particles activated with aminopropyltrimethoxysilane were treated with starch labeled with fluorescein ethylene diamine to produce a solid phase substrate for α-amylase (e.g., TERMAMYL™ or the like).

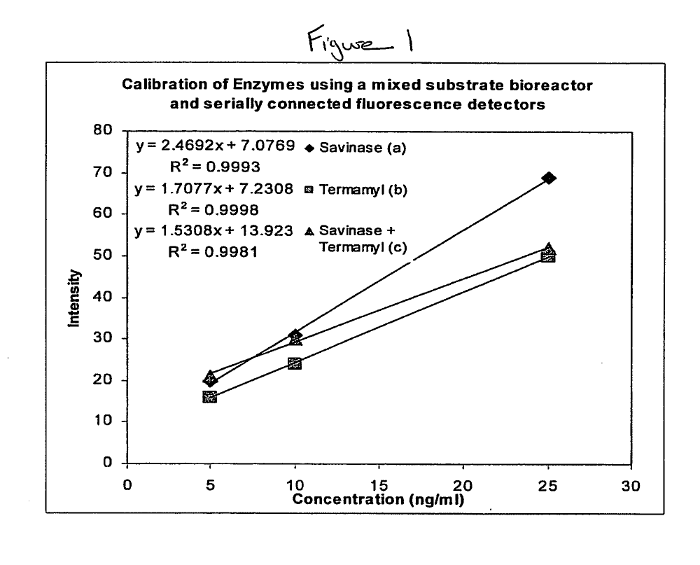

Equal masses of the two solid phase reagents were layered and then packed into a mini-column such that the bottom layer includes the particles coated with the substrate for the subtilisin-type enzyme and the upper layer included the particles coated with the substrate for α-amylase. The mini-column was fed, at a rate of flow of approximately 0.7 ml/min, with samples containing approximately 5 to approximately 25 ng/ml of mixtures of SAVINASE™ and amylase or the individual enzyme component, in phosphate buffered saline (pH 7.4) with Tween 20 (0.1%). The mini-column was linked downstream to two spectrofluorimeters connected in series, the first set at fluorescein fluorescence (approximately 490 nm excitation, and approximately 535 nm emission) and the second at TEXAS RED™ fluorescence (approximately 590 nm excitation, and approximately 620 nm emission), and the fluorescence intensities of the two fluorophores simultaneously and continuously monitored. A response time of approximately 2 minutes was observed for either enzyme with sample repeats possible approximately every 5 minutes. Linear signal/concentration standard curves were obtained for the single enzymes (see FIG. 1A, protease only; FIG. 1B, amylase only), and for the mixture of the two enzymes over this concentration range (FIG. 1C, response for amylase shown) when injected into the column.

It will be appreciated by those of ordinary skill in the art that the present invention can be embodied in various specific forms without departing from the spirit or essential characteristics thereof. The presently disclosed embodiments are considered in all respects to be illustrative and not restrictive. The scope of the invention is indicated by the appended claims, rather than the foregoing description, and all changes that come within the meaning and range of equivalence thereof are intended to be embraced.

All United States patents and applications, foreign and international patents and application, and publications discussed above are hereby incorporated herein by reference in their entireties.

What is claimed is:

1. A method for simultaneously detecting a presence of at least two enzymes in a sample, comprising the steps of:
a.) providing a first substrate for a first enzyme, the first substrate being labeled with a first fluorophore;
b.) providing a second substrate for a second enzyme, the second substrate being labeled with a second fluorophore;
c.) exposing the labeled substrates to the sample to allow the first and second enzymes present in the sample to interact with respective first and second fluorophore-labeled substrates to form respective first and second fluorophore-labeled substrate fragments; and
d.) detecting the presence of the fluorophore-labeled substrate fragments.

2. The method of claim 1, wherein at least one of the first and second substrates comprises one of a protein and a polypeptide.

3. The method of claim 2, wherein the one of the protein and the polypeptide is selected from the group consisting of gelatin, porcine thyroglobulin, collagen, immunoglobulin and bovine serum albumin.

4. The method of claim 1, wherein at least one of the first and second substrates is carried on a support.

5. The method of claim 4, wherein the support comprises a solid phase support, and
wherein the solid phase support comprises one of glass, sol gel beads, and cellulose fibers.

6. The method of claim 5, wherein the sol gel beads are magnetizable.

7. The method of claim 1, wherein at least one of the first and second enzyme is selected from the enzyme group consisting of: protease, cellulase, lipase, α-amylase and collagenase.

8. The method of claim 7, wherein the protease is selected from the group consisting of: subtilisin-type, trypsin, papain, esperase and alcalase.

9. The method of claim 1, wherein at least one of the first and second fluorophore comprises one of fluorescein, rhodamine, TEXAS RED™ and lucifer yellow.

10. The method of claim 1, wherein the sample comprises air.

11. The method of claim 1, wherein enzymes derived from the sample simultaneously contact the first substrate and the second substrate.

12. The method of claim 1, wherein enzymes derived from the sample sequentially contact the first substrate and the second substrate.

13. The method of claim 12, wherein the second substrate comprises a substrate for a protease.

14. A vessel for use in simultaneous detection of a presence of at least two enzymes in a sample, comprising:
a first substrate for a first enzyme, the first substrate being labeled with a first fluorophore; and
a second substrate for a second enzyme, the second substrate being labeled with a second fluorophore.

15. The vessel of claim 14, wherein at least one of the first and second substrates is carried on a support.

16. The vessel of claim 15, wherein the support comprises a solid phase support, and
wherein the solid phase support comprises one of glass, sol gel beads and cellulose fibers.

17. The vessel of claim 16, wherein the vessel comprises a heterogeneous mixture of the first and second substrates.

18. The vessel of claim 16, wherein the vessel comprises a layer of the first substrate and a layer of the second substrate.

19. An apparatus for use in simultaneous detection of at least two enzymes in a sample, comprising:
a vessel,
wherein the vessel comprises:
a first substrate for a first enzyme, the substrate being labeled with a first fluorophore; and
a second substrate for a second enzyme, the second substrate being labeled with a second fluorophore; and
detecting means for detecting fluorescent substrate fragments.

20. The apparatus of claim 19, wherein the first and second substrates are layered within the vessel, and
wherein the layer of the second substrate comprises a substrate for a protease and is located adjacent to the detecting means.

21. An apparatus for use in simultaneous detection of at least two enzymes in a sample, comprising:
a first vessel,
wherein the first vessel comprises:
a first substrate for a first enzyme, the substrate being labeled with a first fluorophore,
wherein the first vessel is connectable to a second vessel, and
wherein the second vessel comprises:
a second substrate for a second enzyme, the substrate being labeled with a second fluorophore; and
detecting means for detecting fluorescent substrate fragments.

22. The apparatus of claim 21, wherein the second substrate in the second vessel comprises a protease, and
wherein the second vessel is located adjacent to the detecting means.
